(19) United States
(12) Patent Application Publication
(10) Pub. No.: US 2021/0154383 A1
(43) Pub. Date: May 27, 2021

(54) SINGLE-USE BREASTMILK COLLECTION DEVICE AND RELATED SYSTEM AND METHOD

(71) Applicant: Jill Gleason, Snohomish, WA (US)

(72) Inventor: Jill Gleason, Snohomish, WA (US)

(21) Appl. No.: 17/105,318

(22) Filed: Nov. 25, 2020

Related U.S. Application Data
(60) Provisional application No. 62/940,140, filed on Nov. 25, 2019.

(51) Int. Cl. A61M 1/06; A61M 1/00

(52) U.S. Cl. CPC A61M 1/064; A61M 1/0031

(57) ABSTRACT

In an embodiment, a breastmilk-extraction-assembly kit includes a flange, a hub, and a check valve. The flange is configured to collapse for packaging, to expand in response to being unpackaged, and to form a seal with a human breast while expanded. The hub has an output port and is configured for coupling to the flange. And the check valve is configured to allow breastmilk to flow out of the hub through the output port during a low-suction portion of a pump cycle, and to impede breastmilk from flowing out of the hub through the output port during a high-suction portion of the pump cycle. Furthermore, the breastmilk-extraction-assembly kit can be disposed in a sealed and size-(e.g., volume-and-dimension)-efficient package for transport, sale, and storage.

### CROSS-REFERENCE TO RELATED APPLICATIONS

This Application claims priority from U.S. Provisional Patent Application Ser. No. 62/940,140 filed Nov. 25, 2019, entitled “SINGLE USE BREAST-MILK COLLECTION DEVICE AND RELATED SYSTEM AND METHOD”; the contents of which are hereby incorporated in its entirety.

### BACKGROUND

A breast pump is a mechanical device that lactating women use to extract milk from one or both of their breasts. Reasons that a woman would use a breast pump include periodically being separated from, and needing another person to feed breast milk to, her breastfeeding infant at feeding time (e.g., while the woman is at work), maintaining or increasing her breast-milk production, and building a reserve supply of breast milk for use when her breasts do not produce enough breast milk for her infant at feeding time. Additionally, many women use breast pumps to donate breastmilk to others in need, for example, to mothers who cannot breastfeed their children.

A breast pump can be a manual device powered from hand or foot movements or an electric device powered from batteries or from electricity via a power outlet. An electric breast pump includes a fluid pump that is powered by a motor to generate, produce, provide, or supply a suction through plastic tubing to a flange that fits over the nipple of a woman's breast. In more detail, the pump achieves a letdown of breast milk by using suction to pull the nipple into the tunnel of a breast shield or flange during a high-suction portion of a milking cycle, and then by releasing the suction, and, therefore, the nipple, during a low-suction portion of the milking cycle; the pump is configured to cycle the high-suction and low-suction portions in a manner that mimics the suckling of an infant. This high-to-low-and-low-to-high suction milking cycle is repeated, for example, approximately 30-60 times per minute, or approximately one time every 1-2 seconds.

The portions of the breast-pump that come into direct contact with the expressed milk and nipple typically must be cleaned after every use to prevent contamination of breastmilk pumped during a subsequent breast-milk pumping session.

For example, the Center for Disease Control (CDC) issued guidelines in 2017 for how to wash such breast-pump components. The process includes using a separate, dedicated wash basin, warm soapy water, sterile drying towels, and sanitizing the components at least once daily (e.g., in a boiling water bath).

### SUMMARY

It follows, therefore, that lactating mothers in the workplace and elsewhere would save time, would reduce the risk of contaminating their breast milk, and, therefore, would reduce the risk of their breast-feeding infants becoming ill due to contaminated breast milk, if there were a breast-milk collection system that required little or no clean-up yet ensured a sterile collection of breast milk every time.

Therefore, a need exists for a partially or fully disposable single-use breastmilk-collection assembly that requires no washing of the assembly components or parts of the breast pump before or after pumping breast milk.

The details of one or more embodiments are set forth in the description below. The features illustrated or described in connection with one exemplary embodiment may be combined with the features of other embodiments. Thus, any of the various embodiments described herein can be combined to provide further embodiments. Aspects of the embodiments can be modified, if necessary to employ concepts of the various patents, applications and publications as identified herein to provide yet further embodiments.

In one embodiment, a breastmilk-extraction-assembly kit is provided. The kit includes a flange configured to collapse for packaging, to expand in response to being unpackaged, and to form a seal with a human breast while expanded; a hub having an output port and configured for coupling to the flange; and a check valve configured to allow breastmilk to flow out of the hub through the output port during a low-suction portion of a pump cycle, and to impede breastmilk from flowing out of the hub through the output port during a high-suction portion of the pump cycle.

### DRAWINGS

Exemplary features of the present disclosure, its nature and various advantages will be apparent from the accompanying drawings and the following detailed description of various embodiments. Non-limiting and non-exhaustive embodiments are described with reference to the accompanying drawings, wherein like labels or reference numbers refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. One or more embodiments are described hereinafter with reference to the accompanying drawings as briefly described below, in which:

FIG. 1 is a diagram showing a woman pumping breast milk with a breastmilk extraction system with disposable components, according to an embodiment;

FIG. 2 is an isometric view of a breastmilk-extraction assembly with a disposable flange, hub, and container according to an embodiment;

FIG. 3 is an exploded view of breastmilk-extraction assembly including a flange, hub, check valve, and container, according to an embodiment;

FIG. 4 is a transparent isometric view of a packaged breastmilk-extraction assembly, according to an embodiment;

FIG. 5 is an isometric view of a container configured to store breastmilk, according to an embodiment;

FIG. 6 is an isometric view with portions broken away of a breast-milk-extraction system and indications of air and breastmilk flow through the system during operation, according to an embodiment;

FIG. 7 is an isometric view of an assembled breast-milk-extraction assembly including a perforated container and disposable hub, valve, and flange portions, according to an embodiment;

FIGS. 8A and 8B are isometric views of a flange in an expanded and in a collapsed configuration, respectively, according to an embodiment;

FIG. 9 is an isometric view of a duckbill check valve, according to an embodiment;

FIG. 10 is an exploded isometric view of a flap check valve, according to an embodiment;

FIG. 11 is an exploded isometric view of a flange, hub, flap check valve, and container of a breastmilk-extraction assembly, according to an embodiment; and

FIG. 12 is an isometric view of the hub of FIGS. 1-4, 6-7, and 11, according to an embodiment.

In accordance with common practice, the various described features are not drawn to scale but are drawn to emphasize specific features relevant to the exemplary embodiments.

### DETAILED DESCRIPTION

In the following detailed description, reference is made to the accompanying drawings that form a part hereof, and in which is shown by way of illustration specific illustrative embodiments. However, it is to be understood that other embodiments may be utilized and that logical, mechanical, and electrical changes may be made. Furthermore, the method presented in the drawing figures and the specification is not to be construed as limiting the order in which the individual steps may be performed. The following detailed description is, therefore, not to be taken in a limiting sense.

Conventional breast-pump assemblies typically require the user to clean (and sometimes even to sterilize) multiple reusable pump components after each use. The recommendations issued by the Center for Disease Control for cleaning breast-pump components are often laborious (e.g., sterilizing by immersing components in boiling water) even in the home, and are also often impractical outside the home.

Consequently, an embodiment of a breastmilk collection device disclosed herein allows lactating women to pump breast milk with no need to wash reusable pump components. This can be a major convenience, especially in workplaces, restaurants, sports venues, and other public locations where the facilities for breast pumping are inadequate (if they even exist at all). The breastmilk collection device provides for sterile pump components every use without the inconvenience of washing or sterilizing components, and potentially with a reduced risk of bacterial and other contamination of expressed milk as compared to conventional breast-milk collection devices. An embodiment of the breast-milk collection device can provide a reduced risk of contamination as compared to conventional breast-milk collection devices due to inadequate cleaning and sterilization of such conventional collection devices. And such a reduced risk of contamination is a significant advance in breast-milk collection devices and systems because such contamination can be harmful or even fatal to infants.

Reusable vacuum tubing typically is a component of the user's own breast pump assembly and is used in conjunction with embodiments of a breastmilk-extraction system disclosed herein. However, an alternative embodiment of the breast pump kit comes with a length of vacuum tubing to be used in conjunction with the user's own breast pump assembly. Like the breastmilk-extraction device itself, the vacuum tubing can be disposed of after a single use, thus further reducing both the time needed to clean and prepare the user's breast-pump assembly while also reducing the risk of dangerous contaminants in reusable assemblies. Additionally, the breastmilk collection device can be compactly packaged and easily assembled and disassembled, which allows the user to easily carry one or multiple disposable devices at a time.

Although the embodiments described below illustrate a breastmilk collection device as applied to a human breast, the techniques described herein may apply to other mammalian breasts where applicable.

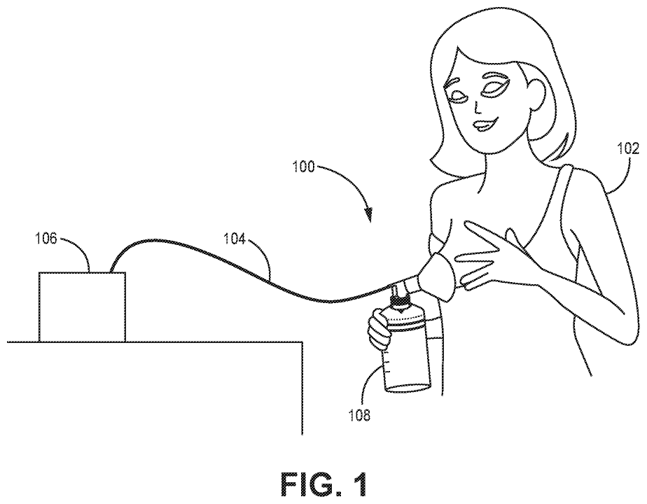

FIG. 1 is a diagram of a woman 102 using an embodiment of a breastmilk collection device 100 for pumping breastmilk, according to an embodiment. The breastmilk collection device 100 is designed so that it can be used once and then disposed of to eliminate the need to clean (e.g., sterilize) and each component before reusing the device. As described in further detail below, the breastmilk collection device 100 can be assembled as a packaged unit that is opened when ready to extract breastmilk, where the package is suitably dimensioned and sized for transport, stocking as inventory, stocking on a store shelf, and storing at one's home.

In some embodiments, the single-use breastmilk collection device 100 is used in conjunction with a breast pump that utilized vacuum tubing 104, according to an embodiment. The breastmilk collection device 100 attaches to the vacuum tubing 104 of a breast pump 106. The breastmilk collection device 100 is held over the nipple of the breast by the user's hand, or may be secured in place by a specially purposed brassiere or other hands-free device, such that the device forms a fluid-tight seal over the nipple of breast. After the pumping of breastmilk is completed, a container 108 is sealed with a screw closure or alternative closure (e.g., a snapping closure seal) and the upper portion of breastmilk collection device 100, including the check valve, hub, and flange, are disposed of in the garbage or recycled. The container 108 containing the extracted breastmilk can then be sealed and stored flat or upright for later use. When container 108 is filled with breastmilk it can be stored upright or, alternatively, the breastmilk can be transferred to another container for storage and container 108 can be disposed of in the garbage or recycling.

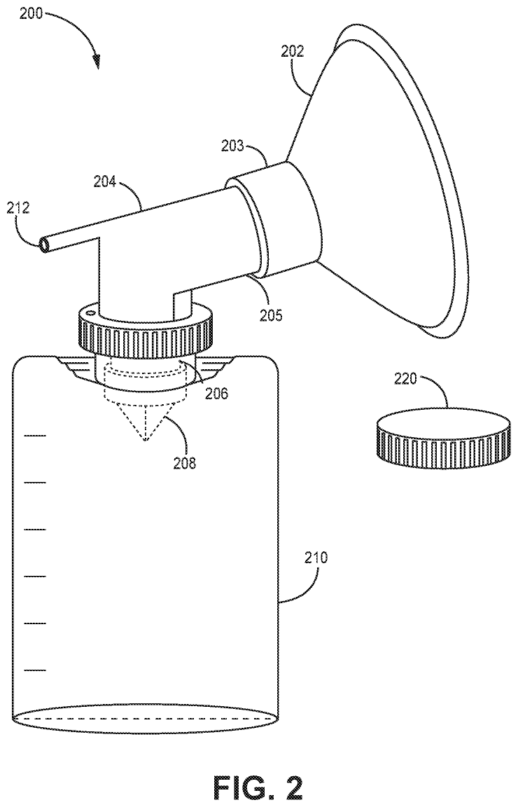

FIG. 2 is a diagram of an embodiment of the breastmilk collection device 200 of FIG. 1. A flange 202 is configured to be held against a breast to create an airtight seal around a nipple of the breast. The flange 202 is configured for mounting to a hub 204 for example, by sliding the flange stem 203 over the input port 205 of hub 204, which can be tapered in some embodiments. However, the flange stem 203 can be configured to slide into, instead of over, the input port 205. The hub 204 includes an interior baffle (not visible in FIG. 2) that directs the breastmilk downward into a check valve 208 that also mounts to the hub at an output port 206 of the hub 204. The check valve 208 allows breastmilk to flow into container 210 during a low-suction portion of a pump cycle, but also prevents the back flow of milk and air from the container to the breast pump during a high-suction portion of a breast-pumping or milking cycle. The path of airflow back to the breast pump is directed upward through the hub 204, and out through a vacuum port 212 to which vacuum tubing is connected. A cap 220 can be used to seal the top of container 210.

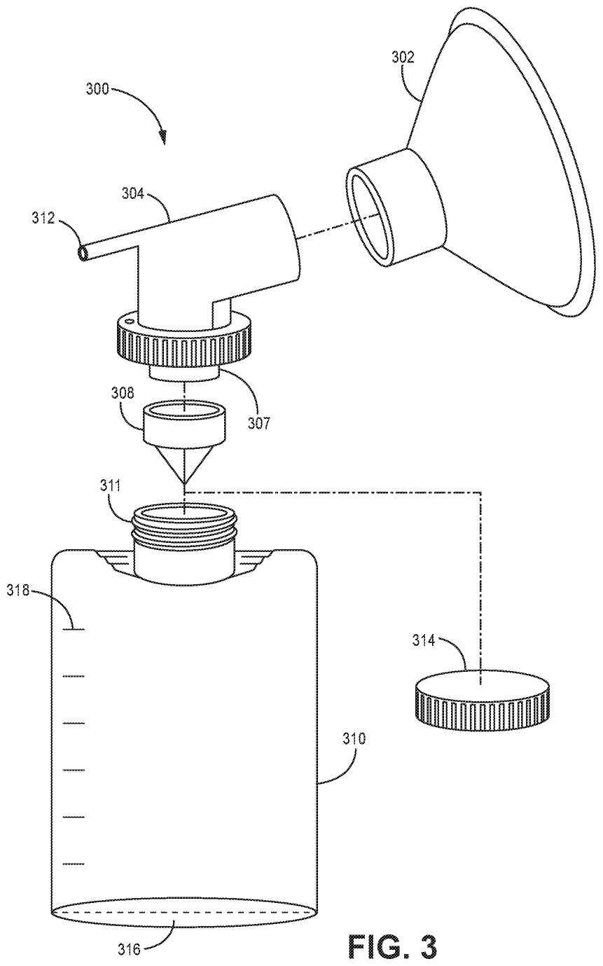

To disassemble the device (as shown in FIG. 3), the user disconnects the vacuum tubing from the vacuum tubing port 312. Container 310 detaches from the upper assembly, which includes the flange 302, hub 304, and check valve 308. In this embodiment, the container 310 has an integrated male screw fitment 311 at a container opening that unscrews from a female screw fitment 307 of the hub 304 and is then sealed by a female screw cap 314. The container 310 may include a gusseted bottom 316 for upright storage, when filled with breast milk or other fluid, if desired. The flange 302, hub 304, and check valve 308 are disposed of in the garbage or are recycled (e.g., by being disposed in a recycling bin or other container) after a breast-pumping session. The container 310 and female screw cap 314 are configured to be used for one-time storage of breastmilk, and for subsequent disposal in the garbage or recycling after use. In some embodiments, container 310 has measurement gradations 318 for indicating an amount of breast milk or other liquid in the container.

The flange 302 is unique in that it is configured to be used once and then disposed of. The flange 302 is made from a flexible material, such as plastic, that can be collapsed before packaging to take up less space for packaging. In an embodiment, the flange 302 is made of flexible, clear polyvinyl chloride. The user (not shown in FIG. 3) then unscrews the female screw fitment 307 and the container 310 containing the collected breastmilk from the upper portion of the breastmilk collection device 300, which includes the flange 302, hub 304, and check valve 308. The upper portion, which, as described above, is configured for a one-time use, may then be disposed of in the garbage or recycling. The user then affixes the female threated screw cap 314 to the male screw fitment 311 to seal the container 310. The user retains the sealed container 310, e.g., in a refrigerator, for later use such as feeding to an infant.

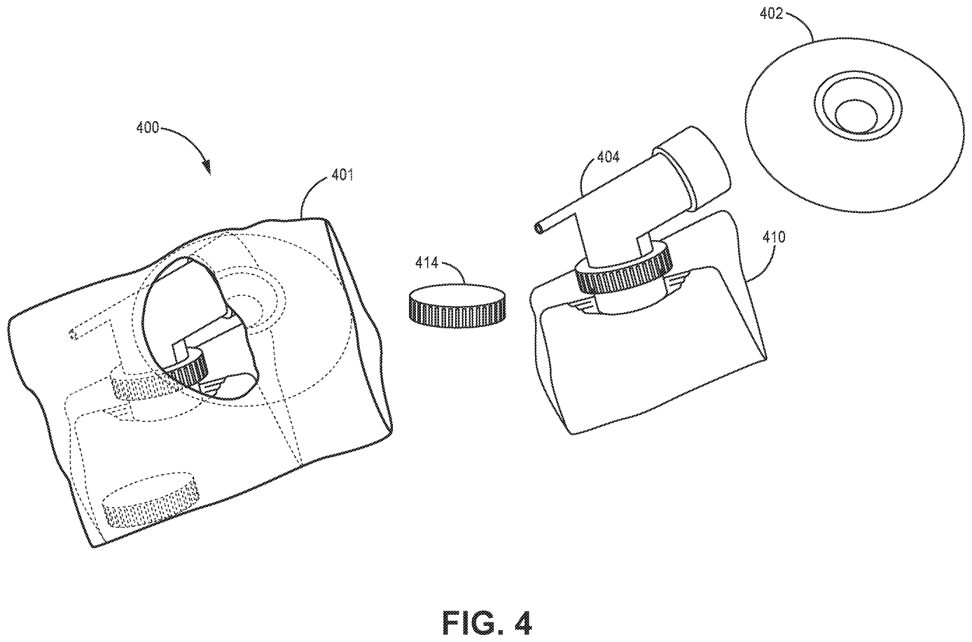

FIG. 4 is an isometric view of one embodiment of the breastmilk-collection-device package or kit 400 disposed within a plastic enclosure 401 for sterile, or at least sealed, contaminant-free transport, sale, and storage. The device components can be arranged separately or can be partially assembled in the packaging 401. For example, in one embodiment, the check valve is already connected to the hub base of hub 404 in the packaging. The container 410 may also be connected to the hub 404. As described above, the packaging enclosure 401 may also include a tubing to connect the hub 404 to a breast pump.

Also, the components can be arranged within the enclosure 401 in a compact fashion to render the package to have a suitably small size for transport and storing as inventory, on a store shelf, and in a home.

In one embodiment, one dimension of the packaged kit 400 is approximately equal to the diameter of the flange 402 in a packaged (compressed) form. Additionally, or alternatively, another dimension of the packaged kit 400 is approximately equal to the width of the container 410. And the depth (thickness) of the packaged kit 400 can be about as thick as a widest thickness of the hub 404. Thus, in one embodiment, the dimensions of the packaged kit 400 are approximately 4.0 inches by 3.0 inches by 0.5 inches, and the volume of the packaged kit is about 6.0 cubic inches.

Achieving, for the packaged kit 400, dimensions that are suitably compact depends, at least in part, on the materials of the breast-milk-extraction-assembly components and how they are arranged in the packaging enclosure 401.

Still referring to FIG. 4, flange 402 is composed of a flexible material that enables it to be collapsible, and indeed is collapsed while in the packaging enclosure 401. To further increase compactness of the packaged kit 400, flange 402 can be folded within the packaging enclosure 401 without irreversibly deforming its shape or damaging its structural integrity. When the packaging enclosure 401 is opened (e.g., by a mother in anticipation of using the device to pump breast milk), flange 402 can be configured to expand to the size and shape shown in FIGS. 1-2 for use during operation of the breastmilk collection device.

In some embodiments, the hub 404 can be positioned adjacent to the flange 402 in the packaging enclosure 401. A portion of flange 402 can rest over the hub 404 to increase the compactness of the packaging enclosure 401. Additionally, the hub 404 and flange 402 can rest on a container 410, which is flattened in packaging enclosure 401. The container 410 can also be folded one or more times while packaged. The female container screw cap 414 is also included in the packaging enclosure, where it can be positioned, for example, under a portion of the flange 402. Thus, in one embodiment, the conical portion of the flange 402 is positioned on top of the hub 404 and both the hub 404 and the flange 402 are positioned on top of the folded container 410 in the packaging 401. The female container screw cap 414 can also be positioned on top of the folded container 410 in the packaging 401. However, the components can be arranged in other ways as well.

Still referring to FIG. 4, packaging of the breastmilk collection device can be achieved as follows, according to an embodiment. A container 410 made of a flexible material can form the base of the packaging assembly, in which the container 410 can be folded one or more times. A hub 404 with a check valve connected at the hub base can be connected to the container 410 and folded near the upper portion of the container 410 so that the hub 404 rests on top of the container 410. Then, a flange 402 can be collapsed and positioned over the container and adjacent to the hub, with the flange stem resting on the container. A portion of the flange 402 can be placed over the hub 404 and/or a female screw cap 414, which may also rest above the folded container 410. The device component assembly can then be wrapped or otherwise enclosed in a plastic wrapping, such as the enclosure 401, and sealed to preserve sterility of the components.

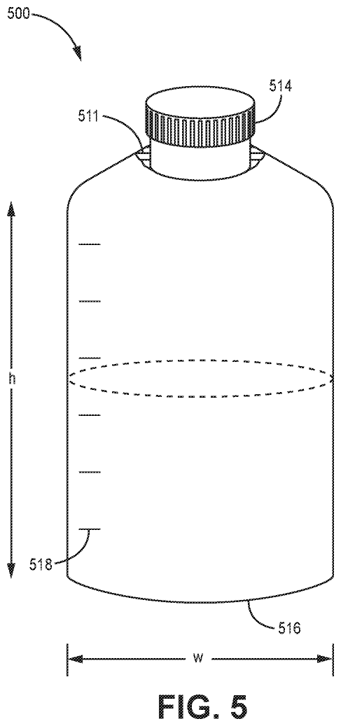

FIG. 5 is an isometric view of one embodiment of a container 500 used in collecting and storing breastmilk and which may be included as part of the breastmilk collection kit 400 of FIG. 4. In some embodiments, container 500 has a width w of approximately 100 mm and a height h of approximately 120 mm. Container 500 may also include a thermal seal portion that is configured to insulate the contents of the container 500 from external heat flow. In an embodiment, the thermal seal portion has a thickness of approximately 6.75 mm around the perimeter of the container 500.

Container 500 is made of a flexible material, e.g., a plastic or plastic polymer material, that is easily foldable without losing structural integrity. The male fitment 511 is fused with the container 500 to create a spouted plastic pouch. In an embodiment, the container 500 is unscrewed from the upper portion of the device (e.g., the hub and the check valve of FIGS. 2-3) and sealed with a female screw cap 514. Furthermore, container 500 can include a gusseted base 516 for upright storage when filled, at least partially, with breast milk or another liquid, and measurement gradations 518 that are etched along the perimeter of the container 500. Once unscrewed from the hub and sealed, the filled container 500 can be stored upright, and after use, can be discarded or recycled similarly to the hub 404, flange 402, and the check valve.

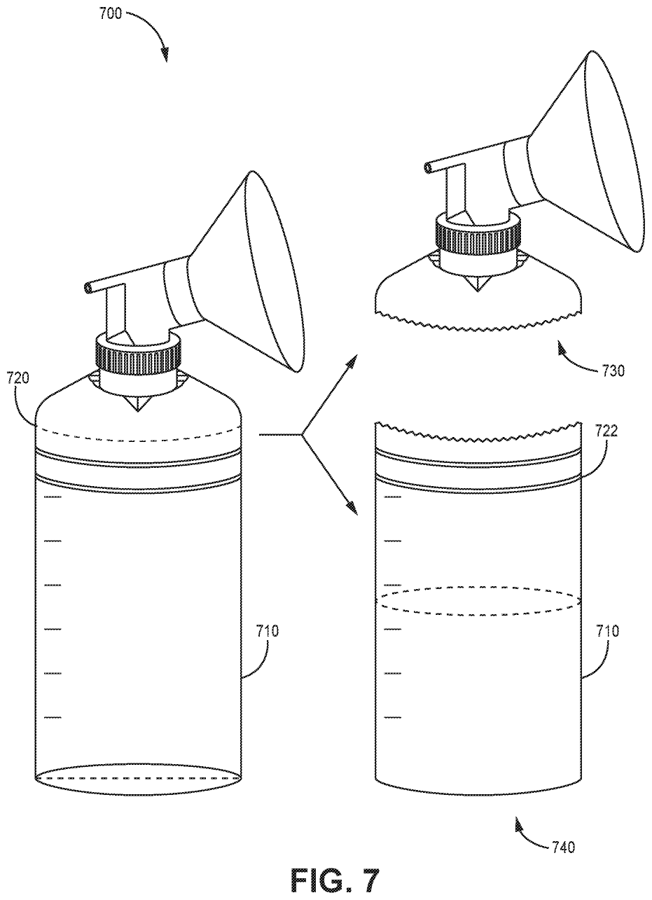

In some embodiments, the container 500 can include a perforated section, as shown in FIG. 7. Prior to a pumping session, the breastmilk collection assembly 700 can be assembled by connecting the hub base to the container 710, or can come pre-assembled in the packaging enclosure 401 described in FIG. 4. Referring to FIG. 7, instead of disassembling the hub 404 from the container 710 after a pumping cycle has been completed (e.g., by unscrewing the female screw fitment 307), however, a user can tear the perforated section 720, thereby separating the upper portion 730 of the device 700 (including the flange, hub, check valve, and a portion of the container 710) from the remaining lower portion 740 of the container 710. The upper portion 730 can then be disposed of or recycled without further disassembly. The perforated section 720 can be positioned anywhere on container 710, but, in an embodiment, is positioned near the top of the container 710 to increase the volume of the remaining lower portion 740 with the collected breastmilk. The container 710 further includes an interlocking closing seal 722 below the perforated section 720, which seal has two interlocking members that can be pressed together to seal the lower portion 740 of the container 710 containing the collected breastmilk, and which can be the same as, or similar too, a seal such as found in Ziploc® plastic storage bags. Including the interlocking closing seal 722 on the container 710 removes the need for a separate female screw cap, which may further increase the compactness of the packaging assembly in FIG. 4.

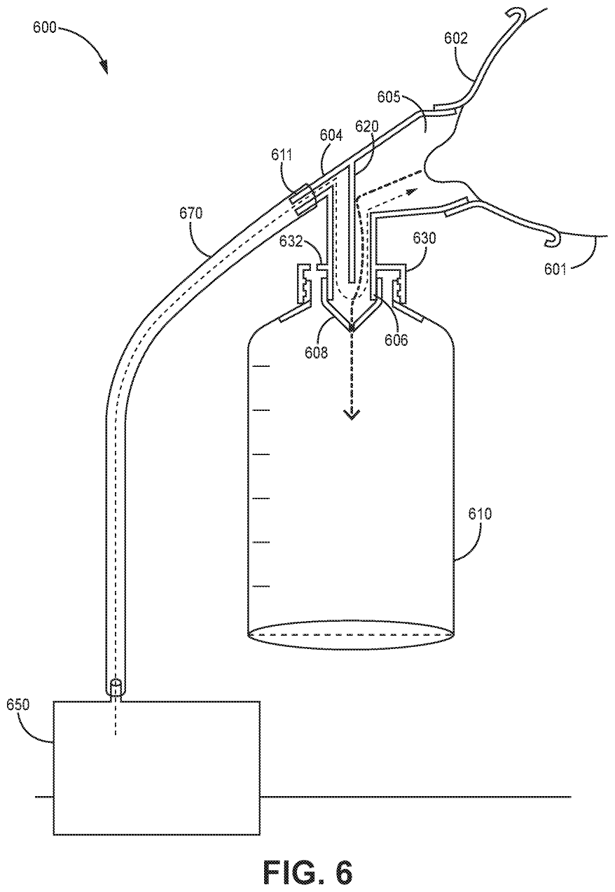

Referring back to FIG. 6, a cross-section of the hub 604 is illustrated in greater detail, along with an example of operation of a breastmilk extraction system 600, according to an embodiment. The breast-pump-suction tubing 670 slides over the tapered vacuum tubing port 611 to create a snug and airtight seal without the use of sealants or adhesives. The hub base includes, at an output port 606, a female screw fitment to accept a male screw fitment from a container 610 and a knurled exterior to facilitate assembly and disassembly of the screw interface by making it easier to grip. Centered within the female screw fitment 4 is an output port 606 to which the check valve 608 mounts.

The hub 604 also includes an interior baffle 620 that terminates just above the hub base, which deflects the extracted breastmilk and directs it downward into the check valve 608. The hub 604 may include a plastic structural support 630 on the exterior of the vacuum chamber 632 for additional structural integrity to the vacuum chamber 632 and input port 605. The vacuum chamber 632 acts as a vent to equalize interior pressure within the container 610 with atmospheric pressure in order to prevent build-up of pressure within the container 610.

FIG. 6 also illustrates the flow of air and breastmilk during a pump cycle. During a high-suction portion of a pump cycle, or when the breast pump 650 increases the suction of air flow through tubing 670 toward the pump 650, the reduced, or more-negative, pressure in the flange 602 tightens and stimulates the breast 601 to secrete breastmilk that is directed toward the interior baffle 620 of hub 604. Air flows through hub 604 and through tubing 670 as directed by the breast pump 650. The extracted breastmilk, however, then travels downward through hub 604 to reach the hub base, which connects to a check valve 608. During the high-suction portion of the pump cycle, the lower (or more-negative) pressure causes the check valve 608 to be closed so that, at least ideally, no milk flows through the check valve 608 and into the container 610, and, likewise, so that no breastmilk is sucked out of the container 610; breastmilk coating the sealing surfaces of the check valve may enhance the seal formed by the check valve while the check valve is closed.

During a low-suction portion of the pump cycle, the breast pump 650 reduces the amount of suction through tubing 670 that is applied to hub 604, though even in this stage the breast pump 650 still creates sufficient suction to maintain the seal between the flange 602 and breast 601. In this phase, if there is any extracted breastmilk, then the extracted breastmilk can still flow through the hub 604 to the hub base, albeit perhaps at a slower flow rate. Once the breastmilk reaches the hub base it comes into contact with check valve 608. Check valve 608 is a one-way valve that allows the breastmilk to collect in the container 610, and also reduces or prevents the backflow of breastmilk up through the hub 604, particularly during the high suction portion of a pump cycle. When in the high suction portion of a pump cycle, the check valve 608 closes to maintain suction within the hub 604 and the flange 602. When suction is relaxed during the low-suction portion of the pump cycle, the breastmilk is able to flow downward through the check valve 608 and into the container 610. This high-low suction cycle is repeated, for example, approximately 30-60 times per minute, or approximately one time every 1-2 seconds, to stimulate a child's suckling. The user holds the device to the breast 601 (or the pump suction alone may be sufficient to maintain the flange 602 against the breast in both the high-suction and low-suction phases of the pump cycle) for the desired length of time, for example 5-20 minutes, or until a desired volume of milk has been expressed, for example, 6 fluid ounces.

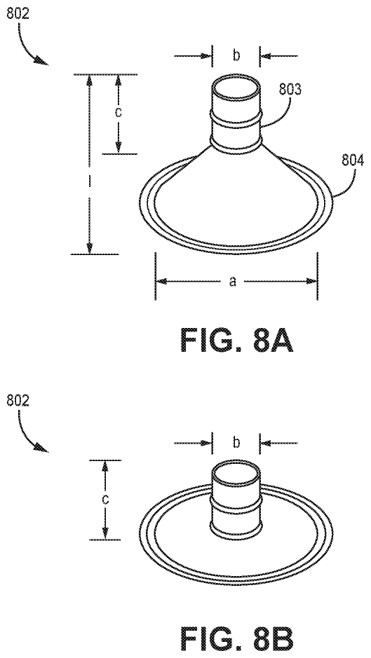

FIGS. 8A and 8B illustrate isometric views of a flange in expanded and collapsed (compressed) states, respectively, according to an embodiment. FIG. 8A depicts a flange 802 while expanded (e.g., during operation of the breastmilk collection device and otherwise any time that the flange is not packaged) while FIG. 8B depicts the flange 802 in a compressed or collapsed position (e.g., while the flange is packaged). Flange 802 is configured to adjust between the expanded and collapsed positions. Flange stem 803 connects to an input port of the hub (see FIGS. 2-3 and 6) by inserting flange stem 803 into the input port to create a tight and snug fit. Conical portion 804 is configured to couple to a human breast, and to form a seal with the breast while expanded during operation of a breastmilk collection system. In some embodiments, flange stem 803 is composed of a flexible material which enables it to collapse.

As shown in FIG. 8A, flange 802 can be conical or funnel-shaped. The outer flange portion 804 has a diameter a which, in some embodiments, can be approximately 82 mm. The flange stem 803 has a diameter b which can be approximately 24.35 mm and a length c of 36 mm. The total length/of the outer flange portion 804 and the stem base 803 when the flange 802 is expanded is, in an embodiment, 68.50 mm. When the flange 802 is collapsed, the length be approximately a length of c, or 36 mm in an embodiment. In some embodiments, the flange 802 is composed of a flexible material (e.g., polyvinyl chloride); however, other materials can also be used.

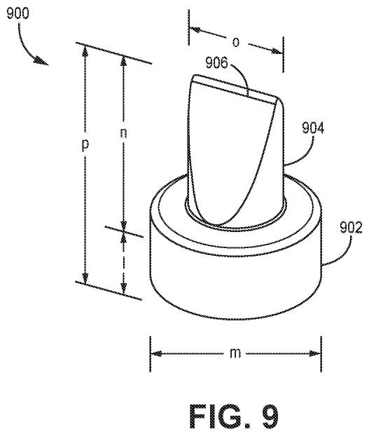

FIG. 9 illustrates a check valve 900 configured to connect with an output port of the hub, according to an embodiment. The check valve 900 is a one-way valve with a cylindrical-shaped first portion 902 and a duckbill-shaped second portion 904. The cylindrical-shaped first portion 902 can have a length/of approximately 11.25 mm and a width m of approximately 22.50 mm. Additionally, the duckbill-shaped second portion 904 can have a length n of approximately 17.75 mm with a diameter o of 12.50 mm. A total length p of the check valve 900 can be 22.50 mm.

The cylindrical-shaped first portion 902 acts as a sleeve that fits snugly over the output port of the hub base. The duckbill-shaped second portion 904 includes an opening 906, in the form of a slit, which enables breastmilk to flow from the cylindrical-shaped first portion 902 out of the duckbill-shaped second portion 904 during a low-suction portion of a pumping cycle, and which prevents breastmilk form flowing out of the container and toward or into the breast pump during a high-suction portion of the pumping cycle.

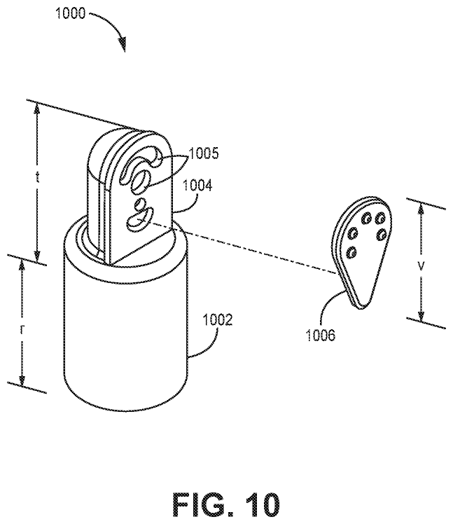

Other mechanical vacuum or gravity regulated one-way valves may be used in alternative embodiments of the check valve, such as that described in FIG. 10. Check valve 1000 includes a cylindrical-shaped first portion 1002 and an elongated second portion 1004, where the check valve 1000 functionally operates similarly to check valve 900. The cylindrical-shaped first portion 1002 is configured to connect to the output port of the hub base. In some embodiments, the cylindrical-shaped first portion 1002 has a length r of, for example, approximately 28.58 mm and a diameter of approximately 15 mm. The elongated second portion 1004 also has a length t which can be approximately 21.75 mm and a diameter of approximately 10 mm including the flap 1006.

During operation of the breastmilk collection device, breastmilk flows from the cylindrical-shaped first portion 1002 down to the elongated second portion 1004, which includes an output port 1005, and which the elongated second portion 1004 is further coupled to flap 1006. In some embodiments, the flap 1006 can have a length v of approximately 21.75 mm. During a high-suction portion of a pump cycle, a suction (e.g., a negative-pressure gradient) within the check valve 1000 causes the flap 1006 to press against the output port 1005 and create a seal, thereby preventing breastmilk from dripping out of outport port 1005 and into a container (as described above, breastmilk coating the sealing surfaces of the output port and the flap may enhance the seal formed by the check valve). When the pump cycle enters a low-suction portion (e.g., a lower negative pressure, zero pressure, or slightly positive pressure), the seal between the flap 1006 and the output port 1005 opens (even ever so slightly), thus enabling breastmilk to flow out of the check-valve output port 1005 and into the container.

The flap 1006 can be made out of a Silicon polymer or other Silicon-based material. In some embodiments the flap forms a teardrop shape as shown in FIG. 10.

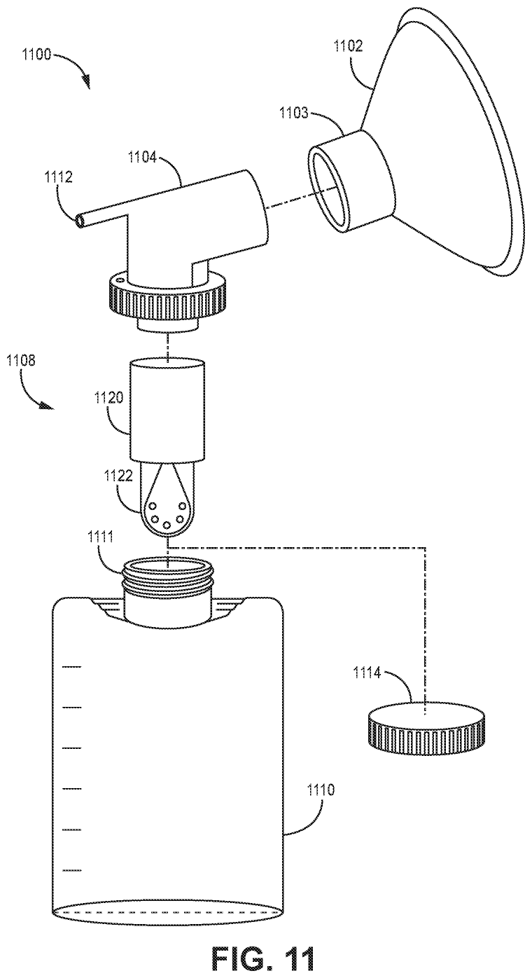

FIG. 11 is an exploded view of the breast-milk extraction assembly 1100 including the check valve 1000 of FIG. 10, according to an embodiment. The flange stem 1103 fits inside the input port of hub 1104. Vacuum tubing (not shown in FIG. 11), which may be provided with the breast-milk collection assembly 1100 or separately acquired, is connected to vacuum tubing port 1112 and connected to a breast pump (not shown in FIG. 11). The cylindrical-shaped first portion 1120 of the check valve 1108 fits snugly within or around the output port of hub 1104. The elongated second portion 1122 can be inserted through the male screw fitment 1111 into container 1110. After use, the upper assembly including the flange 1102, hub 1104, and check valve 1108 can be disconnected from container 1110 and the female screw cap 1114 can be fastened on the male screw fitment 1111 to seal the container 1110.

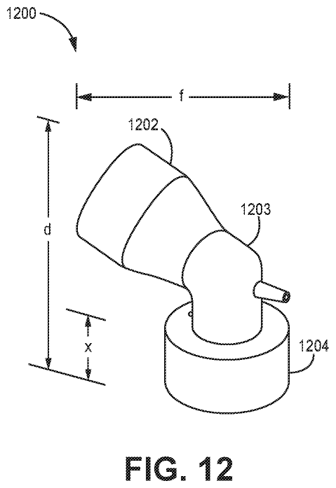

FIG. 12 illustrates an isometric view of the hub 1200, according to an embodiment. Hub 1200 has a length d which in some embodiments, is approximately 61.11 mm and a width e of about 57.16 mm. The hub base 1204 has a length f which can be about 16.35 mm and a width g of approximately 32 mm. In some embodiments the input port 1202 of hub 1200 can be a tapered receptacle that tapers gradually from the input port 1202 (where a flange stem is inserted) to the hub center 1203 of hub 1200. The hub base 1204 can have a length x of 16.35 mm.

The terms “about” or “approximately” mean that the value or parameter specified may be somewhat altered, as long as the alteration does not result in nonconformance of the process or structure to the illustrated embodiment from the perspective of one having ordinary skill in the art. For instance, unless otherwise indicated, a numerical quantity modified by the term “approximately” can be altered to within ±20% of the specified value. Finally, the term “exemplary” merely indicates the accompanying description is used as an example, rather than implying an ideal, essential, or preferable feature of the invention.

### Example Embodiments

Example 1 includes a breastmilk-extraction-assembly kit, comprising: a flange configured to collapse for packaging, to expand in response to being unpackaged, and to form a seal with a human breast while expanded; a hub having an output port and configured for coupling to the flange; and a check valve configured to allow breastmilk to flow out of the hub through the output port during a low-suction portion of a pump cycle, and to impede breastmilk from flowing out of the hub through the output port during a high-suction portion of the pump cycle.

Example 2 includes the kit of Example 1, comprising a container configured to couple to the hub, composed of a flexible material, and configurable to be flat.

Example 3 includes the kit of Example 2, wherein the container: is perforated around a perimeter of the container; and comprises a closing seal around the perimeter.

Example 4 includes the kit of any of Examples 2-3, wherein the container comprises measurement gradations etched along the perimeter.

Example 5 includes the kit of any of Examples 1-4, wherein the flange is composed of polyvinyl chloride.

Example 6 includes the kit of any of Examples 1-5, wherein the hub comprises: an interior baffle configured to deflect the breastmilk towards the check valve; a hub base configured to mechanically couple the hub to a container; and a vacuum chamber configured to equalize pressure inside and outside of the container.

Example 7 includes the kit of any of Examples 1-6, wherein the check valve comprises: a cylindrical-shaped portion configured to mechanically couple with the hub; and a duckbill-shaped portion configured to transfer the breastmilk to the container.

Example 8 includes the kit of any of Examples 1-7, wherein the check valve comprises: a cylindrical-shaped first portion configured to mechanically couple with the hub; and a second portion comprising: a second valve configured to receive the breastmilk from the cylindrical-shaped portion, and a flap coupled to the second valve and configured to: separate from the second valve during the low-suction portion of the pumping cycle, and seal with the second valve during the high-suction phase of the pumping cycle.

Example 9 includes the kit of any of Examples 1-8, wherein the check valve comprises a flexible Silicon-based material.

Example 10 includes a system, comprising: a flange configured to collapse for packaging, to expand in response to being unpackaged, and to form a seal with a breast while expanded; a hub configured for coupling to the flange to a container; and a check valve configured to allow breastmilk to flow from the hub to the container during a low-suction portion of a pump cycle, and to form a seal between the hub and the container during a high-suction portion of a pump cycle.

Example 11 includes the system of Example 10, comprising a breast pump coupled to the hub and configured: to produce increased suction to the flange during the high-suction portion of the pumping cycle; and to produce decreased suction to the flange during the low-suction portion of the pumping cycle.

Example 12 includes the system of any of Examples 10-11, further comprising: the container configured to couple to the hub; and wherein the container is composed of a flexible material and is configurable to be flat.

Example 13 includes the system of any of Examples 11-12, further comprising a tubing configured for connecting to the breast pump and the hub and to transfer suction between the breast pump and the hub.

Example 14 includes the system of any of Examples 10-13, wherein the container is perforated around a perimeter of the container, and wherein the container comprises an interlocking closure seal around the perimeter.

Example 15 includes a packaged breastmilk-extraction assembly, comprising: a package; a collapsed flange disposed in the package, configured to expand in response to being unpackaged, and configured to form a seal with a human breast while expanded; a hub disposed in the package adjacent to the collapsed flange, the hub having an output port and configured for coupling to the flange; and a check valve disposed in the package, connected to the hub, and configured to allow breastmilk to flow out of the hub through the output port during a low-suction portion of a pump cycle, and to impede breastmilk from flowing out of the hub through the output port during a high-suction portion of the pump cycle.

Example 16 includes the packaged breastmilk-extraction assembly of Example 15, further comprising a container disposed in the package beneath the collapsed flange and the hub and configured to couple to the hub; and where the container is composed of a flexible material and is flat while enclosed in the packaging film, and is configured to expand in response to being unpackaged.

Example 17 includes the packaged breastmilk-extraction assembly of Example 16, wherein the container: is perforated around a perimeter of the container; and comprises an interlocking closing seal around the perimeter.

Example 18 includes the packaged breastmilk-extraction assembly of any of Examples 15-17, wherein a portion of the collapsed flange is placed over the hub.

Example 19 includes the packaged breastmilk-extraction assembly of any of Examples 15-18, wherein a dimension of the packaged breastmilk-extraction assembly is approximately equal to a diameter of the collapsed flange.

Example 20 includes the packaged breastmilk-extraction assembly of any of Examples 16-19, wherein a dimension of the packaged breastmilk-extraction assembly is approximately equal to a width of the container.

Example 21 includes the packaged breastmilk-extraction assembly of any of Examples 15-20, wherein a depth of the packaged breastmilk-extraction assembly is approximately equal to a depth of the hub.

Example 22 includes the packaged breastmilk-extraction assembly of any of Examples 15-21, wherein a length, width, and depth of the packaged breastmilk-extraction assembly are approximately 4 inches, 3 inches, and Example 0.5 inches, respectively.

Example 23 includes the packaged breastmilk-extraction assembly of any of Examples 15-22, wherein a volume of the packaged breastmilk-extraction assembly is approximately 6 cubic inches.

Example 24 includes a method, comprising: attaching a check valve to a hub, the check valve configured to allow breastmilk to flow out of the hub during an low-suction portion of a pump cycle, and to impede breastmilk from flowing out of the hub during a high-suction portion of the pump cycle; attaching the hub to a flattened container configured to expand; folding the flattened container; positioning a collapsed flange adjacent to the hub, the collapsed flange configured to expand in response to being unpackaged, and to form a seal with a human breast while expanded; and enclosing the check valve, hub, flattened container, and collapsed flange in a package.

Example 25 includes the method of Example 24, wherein positioning the collapsed flange adjacent to the hub comprises positioning the collapsed flange and the hub over the container.

Example 26 includes a method, comprising: forming a seal between a flange and a human breast, the flange being configured to collapse for packaging and to expand in response to being unpackaged; directing, to a hub coupled to the flange and having an output port, breast milk from the human breast during a high-suction portion of a pump cycle; transferring, to a container coupled to the hub, the breastmilk during a low-suction portion of a pump cycle, the container composed of a flexible material and configurable to be flat for packaging; and

Example 27 includes the method of Example 26, further comprising preventing breastmilk from re-entering the output port of the hub during a low-suction portion of the pump cycle.

Example 28 includes the method of any of Examples 26-27, further comprising disposing of the flange, hub, and check valve after transferring breastmilk to the container.

From the foregoing it will be appreciated that, although specific embodiments have been described herein for purposes of illustration, various modifications may be made without deviating from the spirit and scope of the disclosure. Furthermore, where an alternative is disclosed for a particular embodiment, this alternative may also apply to other embodiments even if not specifically stated. Moreover, one or more components of a described apparatus or system, or one or more steps of a described method, may have been omitted from the description for clarity or for another reason. In addition, one or more components of a described apparatus or system that have been included in the description may be omitted from the apparatus or system, and one or more steps of a described method that have been included in the description may be omitted from the method.

What is claimed is:

1. A breastmilk-extraction-assembly kit, comprising:
a flange configured to collapse for packaging, to expand in response to being unpackaged, and to form a seal with a human breast while expanded;
a hub having an output port and configured for coupling to the flange; and
a check valve configured to allow breastmilk to flow out of the hub through the output port during a low-suction portion of a pump cycle, and to impede breastmilk from flowing out of the hub through the output port during a high-suction portion of the pump cycle.

2. The kit of claim 1, comprising a container configured to couple to the hub, composed of a flexible material, and configurable to be flat.

3. The kit of claim 2, wherein the container:
is perforated around a perimeter of the container; and
comprises a closing seal around the perimeter.

4. The kit of claim 2, wherein the container comprises measurement gradations etched along a perimeter of the container.

5. The kit of claim 1, wherein the flange is composed of polyvinyl chloride.

6. The kit of claim 1, wherein the hub comprises:
an interior baffle configured to deflect the breastmilk towards the check valve;
a hub base configured to mechanically couple the hub to a container; and
a vacuum chamber configured to equalize pressure inside and outside of the container.

7. The kit of claim 1, wherein the check valve comprises:
a cylindrical-shaped portion configured to mechanically couple with the hub; and
a duckbill-shaped portion configured to transfer the breastmilk to a container.

8. The kit of claim 1, wherein the check valve comprises:
a cylindrical-shaped first portion configured to mechanically couple with the hub; and
a second portion comprising:
a second valve configured to receive the breastmilk from the cylindrical-shaped portion, and
a flap coupled to the second valve and configured to:
separate from the second valve during the low-suction portion of the pumping cycle, and
seal with the second valve during a high-suction phase of the pumping cycle.

9. The kit of claim 1, wherein the check valve comprises a flexible Silicon-based material.

10. A system, comprising:
a flange configured to collapse for packaging, to expand in response to being unpackaged, and to form a seal with a breast while expanded;
a hub configured for coupling to the flange to a container; and
a check valve configured to allow breastmilk to flow from the hub to the container during a low-suction portion of a pump cycle, and to form a seal between the hub and the container during a high-suction portion of a pump cycle.

11. The system of claim 10, comprising a breast pump coupled to the hub and configured:
to produce increased suction to the flange during the high-suction portion of the pumping cycle; and
to produce decreased suction to the flange during the low-suction portion of the pumping cycle.

12. The system of claim 10, further comprising:
the container configured to couple to the hub; and
wherein the container is composed of a flexible material and is configurable to be flat.

13. The system of claim 11, further comprising a tubing configured for connecting to the breast pump and the hub and to transfer suction between the breast pump and the hub.

14. The system of claim 10, wherein the container is perforated around a perimeter of the container, and wherein the container comprises an interlocking closure seal around the perimeter.

15. A packaged breastmilk-extraction assembly, comprising:
a package;
a collapsed flange disposed in the package, configured to expand in response to being unpackaged, and configured to form a seal with a human breast while expanded;
a hub disposed in the package adjacent to the collapsed flange, the hub having an output port and configured for coupling to the flange; and
a check valve disposed in the package, connected to the hub, and configured to allow breastmilk to flow out of the hub through the output port during a low-suction portion of a pump cycle, and to impede breastmilk from flowing out of the hub through the output port during a high-suction portion of the pump cycle.

16. The packaged breastmilk-extraction assembly of claim 15, further comprising a container disposed in the package beneath the collapsed flange and the hub and configured to couple to the hub; and where the container is composed of a flexible material and is flat while enclosed in the package, and is configured to expand in response to being unpackaged.

17. The packaged breastmilk-extraction assembly of claim 16, wherein the container: is perforated around a perimeter of the container; and comprises an interlocking closing seal around the perimeter.

18. The packaged breastmilk-extraction assembly of claim 15, wherein a portion of the collapsed flange is placed over the hub.

19. The packaged breastmilk-extraction assembly of claim 15, wherein a dimension of the packaged breastmilk-extraction assembly is approximately equal to a diameter of the collapsed flange.

20. The packaged breastmilk-extraction assembly of claim 16, wherein a dimension of the packaged breastmilk-extraction assembly is approximately equal to a width of the container.

21. The packaged breastmilk-extraction assembly of claim 15, wherein a depth of the packaged breastmilk-extraction assembly is approximately equal to a depth of the hub.

22. The packaged breastmilk-extraction assembly of claim 15, wherein a length, width, and depth of the packaged breastmilk-extraction assembly are approximately 4 inches, 3 inches, and 0.5 inches, respectively.

23. The packaged breastmilk-extraction assembly of claim 15, wherein a volume of the packaged breastmilk-extraction assembly is approximately 6 cubic inches.

24. A method, comprising:
attaching a check valve to a hub, the check valve configured to allow breastmilk to flow out of the hub during a low-suction portion of a pump cycle, and to impede breastmilk from flowing out of the hub during a high-suction portion of the pump cycle;
attaching the hub to a flattened container configured to expand;
folding the flattened container;
positioning a collapsed flange adjacent to the hub, the collapsed flange configured to expand in response to being unpackaged, and to form a seal with a human breast while expanded; and
enclosing the check valve, hub, flattened container, and collapsed flange in a package.

25. The method of claim 24, wherein positioning the collapsed flange adjacent to the hub comprises positioning the collapsed flange and the hub over the container.

26. A method, comprising:
forming a seal between a flange and a human breast, the flange being configured to collapse for packaging and to expand in response to being unpackaged;
directing, to a hub coupled to the flange and having an output port, breastmilk from the human breast during a high-suction portion of a pump cycle;
transferring, to a container coupled to the hub, the breastmilk during a low-suction portion of a pump cycle, the container composed of a flexible material and configurable to be flat for packaging; and

27. The method of claim 26, further comprising preventing breastmilk from re-entering the output port of the hub during a high-suction portion of the pump cycle.

28. The method of claim 26, further comprising disposing of the flange, hub, and check valve after transferring breastmilk to the container.
